(19) United States
(12) Patent Application Publication
(10) Pub. No.: US 2026/0199203 A1
(43) Pub. Date: Jul. 16, 2026

(54) MICROPARTICULATE COMPOSITE MATERIAL BASED ON ZINC OXIDE GRAFTED TO DIATOMACEOUS EARTH, METHOD FOR PREPARING SAME AND USES THEREOF AS AN ANTI-UV ACTIVE AGENT

(71) Applicant: VINPAI, SAINT-DOLAY (FR)

(72) Inventors: Hervé DEMAIS, GUIDEL (FR); Philippe LE RAY, PORNICHET (FR)

(21) Appl. No.: 18/872,483

(22) PCT Filed: Jun. 9, 2023
(86) PCT No.: PCT/FR2023/050829

§ 371 Date: Dec. 6, 2024

(30) Foreign Application Priority Data
Jun. 10, 2022 (FR) FR2205610

(51) Int. Cl. A61K 8/27; A61K 8/02; A61K 8/96; A61Q 17/04; C09C 1/30

(52) U.S. Cl. CPC A61K 8/27; A61K 8/0245; A61K 8/965; A61Q 17/04; C09C 1/3018; C09C 1/3027; C09C 1/3054; C09C 1/309; A61K 2800/621; A61K 2800/651; C01P 2004/03; C01P 2004/61; C01P 2004/62; C01P 2004/64; C01P 2004/84

(57) ABSTRACT

The invention relates to a method for preparing a composite material comprising zinc oxide crystals grafted to the surface of diatomaceous earth particles, comprising a step of milling in the presence of an agent for stabilizing the electron charges created during the milling, and also to a composite material comprising zinc oxide crystals grafted to the surface of diatomaceous earth particles, and to the use thereof as an active agent for protection against UV radiation.

### TECHNICAL FIELD

The present invention relates to the field of microparticulate composite materials, notably based on zinc oxide.

### PRIOR ART

It is well known that ultraviolet (UV) radiation, emitted naturally by the sun or by artificial sources, is beneficial to the human body in small amounts, and is essential for the synthesis of vitamin D, which in turn is essential for fixing calcium in the bones and preventing osteoporosis. However, overexposure to UV radiation is responsible for impairing the skin (accelerated aging, cancer, etc.), the eyes and the immune system.

An antisun cream is a cream or lotion used for reducing the exposure of the skin to the sun's ultraviolet rays. As such, it constitutes a means of passive external photoprotection, functioning as an ultraviolet screening agent. Such an antisun product is composed of ultraviolet screening agents in a base which may be an oil or, more frequently, an emulsion (cream or lotion). Effective protection must block both UVA and UVB rays: UVB (and to a lesser extent UVA) may cause sunburn, UVA causes premature skin aging, UVA and especially UVB cause skin cancer.

An ultraviolet screening agent or UV screening agent is a chemical compound that blocks or absorbs ultraviolet radiation. All antisun products contain ultraviolet screening agents, which are divided into two families of molecules: chemical (or organic) screening agents and mineral (or inorganic) sunblocks.

Organic screening agents are easier to use, but they pollute water, are difficult to eliminate, even by sewage treatment plants, may be allergenic and prove to be toxic to both man and the environment, in particular aquatic biotopes.

An alternative to organic screening agents is the use of mineral sunblocks. In cosmetics, only two mineral sunblocks are permitted: titanium dioxide and zinc oxide. These two sunblocks provide immediate UV protection, unlike chemical screening agents which take up to 25 minutes to ensure the claimed protection.

The mineral sunblocks used in “ecologically organic” creams are hypoallergenic and photostable (making them the recommended antisun products for allergy sufferers and children). For a long time, however, they were less appreciated by consumers because they were more difficult to spread and gave users an unattractive whitish tint. To solve this problem and improve the cosmetic aspect, laboratories reduced these mineral sunblocks in the form of nanoparticles to formulate antisun creams. Nevertheless, numerous studies have been conducted on nanoparticles, and have demonstrated their harmful effects on the environment, notably on phytoplankton, and their potential effects on health, as these particles penetrate the skin. In particular, TiO₂ nanoparticles have become the main oxidizing agent entering coastal waters, with direct ecological consequences on the ecosystem. Titanium dioxide nanoparticles disperse in water, finding their way into filter-feeding organisms (notably oysters and mussels) and the cells of other marine animals.

Zinc oxide thus remains a preferred choice due to its many qualities as an efficient, photostable, non-allergenic agent for screening out UVB and UVA radiation. However, in its native nanoparticulate form (particles smaller than 100 nm), it presents a risk to the environment and potentially to human health, due to its size and its oxidizing properties under the action of UV radiation.

In view of these findings concerning the limits and dangers of using organic screening agents in antisun creams, and to take account of the risk of using nanoparticulate mineral sunblocks, it is necessary to find an alternative making it possible to reconcile protection that is both effective for humans and harmless to the environment.

### GENERAL DESCRIPTION

The Applicant has, to its credit, solved this problem by developing a process enabling the production of a composite material comprising zinc oxide nanoparticles grafted onto natural mineral microparticles of diatomaceous earth, said composite material having particle sizes ranging from 30 to 200 nm.

Patent EP 2 375 575 describes a cosmetic composition comprising a composite material based on diatomaceous earth and zinc. This composite material is prepared on the basis of unmodified diatomaceous earth and thus has a mean size of 10 microns, which leads to poor dispersion of the material in a cosmetic composition. Moreover, the Applicant has found that, under the zinc chloride concentration conditions described in said patent, the proposed zinc oxide concentration leads to very partial coverage of the diatomaceous earth particles by the zinc crystals, and that the zinc oxide concentration of the final product is 10 times lower than indicated. As it stands, the preparation method proposed cannot lead to the production of a material that can claim to have the alleged UV-stabilizing properties.

On the contrary, in the composite material according to the invention, modification of diatomaceous earth in the form of microparticles allows a very significant increase in the specific surface area of the material, leading to a very significant increase in the crystallization surface area of zinc oxide. This allows the surface properties to be increased for the same amount of material, in particular the surface area exposed to light rays, including UV rays, and the possibility of diffraction and refraction of these rays. This technique allows the development of large surface areas of zinc nanocrystals while at the same time very strongly limiting the diffusion of the material into the environment, since the zinc oxide nanoparticles are fixed to this non-nanometric support and thus cannot passively diffuse into living cells or organisms.

A first subject of the invention is thus a process for preparing a composite material comprising zinc oxide crystals grafted onto the surface of diatomaceous earth particles, comprising the following steps:
1) preparing diatomaceous earth microparticles by milling diatomaceous earth;
2) grafting zinc salt crystals onto the surface of the diatomaceous earth microparticles;
3) converting said zinc salt crystals grafted onto the surface of the diatomaceous earth microparticles into zinc oxide crystals grafted onto the surface of the diatomaceous earth microparticles;
4) milling the microparticles obtained in step 3) to an individual particle size ranging from 10 to 300 nm, this milling being performed in the presence of an agent for stabilizing the electronic charges created during milling.

A second subject of the invention is a composite material comprising zinc oxide crystals grafted onto the surface of diatomaceous earth particles, said grafted diatomaceous earth particles having an individual particle size ranging from 10 to 300 nm. This composite material is advantageously prepared via the process according to the invention. The invention also relates to the use of the composite material according to the invention or prepared via the process according to the invention as an active agent for protecting against UV radiation, notably in cosmetic compositions.

Another subject of the invention is a cosmetic composition comprising the composite material according to the invention or prepared via the process according to the invention.

### DETAILED DESCRIPTION

A first subject of the invention is a process for preparing a composite material comprising zinc oxide crystals grafted onto the surface of diatomaceous earth particles, comprising the following steps:
1) preparing diatomaceous earth microparticles by milling diatomaceous earth in aqueous medium;
2) grafting zinc salt crystals onto the surface of the diatomaceous earth microparticles;
3) transforming said zinc salt crystals grafted onto the surface of the diatomaceous earth microparticles into zinc oxide crystals grafted onto the surface of the diatomaceous earth microparticles;
4) milling the microparticles obtained in step 3) in aqueous medium to an individual particle size ranging from 10 to 300 nm, this milling being performed in the presence of an agent for stabilizing the electronic charges created during milling.

Diatomaceous earth (also referred to as kieselguhr or diatomite) is a microscopic, unicellular fossilized alga found in large geological deposits in ancient marine sedimentary basins. This sedimentary rock has a silica-based protective envelope, known as a frustule, with a predominantly silicic composition and thus varying in shape and size depending on the age and origin of the sediment. Frustules have a noteworthy structure, as they are composed of a large network of macroscopic channels that can represent up to more than 80% of the structure's total volume.

In step 1) of the process according to the invention, the diatomaceous earth is advantageously milled to an individual particle size ranging from 50 nm to 2 μm, preferably from 100 nm to 1 um and even more preferably from 150 nm to 500 nm. The term “individual particle size” means that each particle of the material has a particle size falling within the indicated range of values. The particle size is determined by scanning electron microscopy (SEM).

Advantageously, the milling in the first step is performed so as to conserve the silanol groups on the surface of the diatomaceous earth. Specifically, the Applicant realized that increasing the temperature of the material excessively leads to amorphization of the silica structure, with loss of the surface silanol groups as a result of dehydroxylation of these groups.

Conservation of a maximum number of surface silanol groups may be achieved via a technique involving milling in an aqueous and sequential medium, allowing the temperature rise during milling to be kept below a threshold of 200° C.

Thus, the milling step 1) of the process according to the invention is advantageously performed in aqueous medium using a planetary-type mill.

This type of mill operates on the following principle: The milling bowl is arranged eccentrically on the sun wheel of the planetary ball mill. The sun wheel rotates in the opposite direction to the milling bowl. The milling balls in the bowl are deflected by overlapping rotational movements, which are responsible for said Coriolis force. The Coriolis force is a force that deflects the trajectory of a moving subject at the surface of a rotating object. It applies in particular to moving masses of air and water. Velocity differences between the balls and the milling bowl result in interaction of the impact and friction forces, releasing significant dynamic energies. The combination of these forces is reflected by the high milling efficiency of planetary ball mills.

This milling step may be performed by varying one or more of the following parameters:
the volume of the milling feedstock, i.e. the volume occupied by the milling balls in relation to the volume of the milling bowl;
the particle size of the milling feedstock (the size of the milling bodies determines the fineness of the output particle size: the smaller the milling balls, the finer the final particle size can be);
the feedstock of product to be milled, and the proportional water feedstock;
the speed of rotation of the mill;
the number of milling cycles;
the temperature of the mixture during milling.

A person skilled in the art can adapt the parameters to obtain the desired particle size. Generally, they will use a volume of milling feedstock of about 15% to 25%. The milling balls are advantageously smaller than 1 mm, preferably 0.5 mm. The number of milling cycles is generally from 1 to 20, preferably from 5 to 15 and even more preferably from 8 to 12. As indicated previously, the temperature during milling is advantageously kept below 200° C., preferably below 150° C. As regards the speed of rotation of the mill, this is advantageously of the order of 300 to 800 rpm, for example 550 rpm.

Step 2) of the process according to the invention may comprise the following steps:
a) placing in aqueous suspension the diatomaceous earth particles obtained in step 1 with calcium carbonate;
b) placing the mixture obtained at the end of step a) in contact with an aqueous suspension of zinc chloride;
c) stirring the mixture obtained at the end of step b) at a temperature ranging from 5° C. to 40° C., preferably 15° C. to 25° C., for a period ranging from 24 to 72 hours, preferably 48 hours;
d) recovering the composite material obtained at the end of step c), notably by sedimentation or centrifugation;
e) drying the composite material at a temperature ranging from 50° C. to 90° C., preferably 70° C., for a period ranging from 12 to 48 hours, preferably 24 hours.

This second process step allows the zinc salts to react with the silanol groups on the surface of the diatomaceous earth particles. In particular, this step allows the calcium carbonate present in the diatomaceous earth to dissolve, releasing hydroxyl and carbonate species into the medium, leading to crystallization of hydrated zinc carbonate hydroxide phases of chemical formula Zn₄(CO₃)(OH)₆·H₂O at the surface of the diatomaceous earth particles on the surface silanol groups.

In particular, the diatomaceous earth particles obtained in step 1 may be suspended in water in a proportion of 0.1 to 100 grams of powder per liter of water, preferably 10 grams of powder per liter of water.

Similarly, calcium carbonate may be suspended in water in a proportion of 0.1 to 100 grams of calcium carbonate per liter of water, preferably 10 grams of calcium carbonate per liter of water.

The aqueous zinc chloride suspension used in step b) has a concentration of 5×10⁻³ to 5 mol.L⁻¹, preferably 2 to 3 mol.L⁻¹, notably about 1 mol.L⁻¹.

Advantageously, the suspension obtained at the end of step a) and the zinc chloride suspension may be hydrated separately for 15 to 60 minutes, preferably 30 minutes, before the two suspensions are placed together in step b).

Step c) of stirring of the mixture may be performed using a paddle mixer at a rotational speed of about 100 rpm.

Step e) of drying of the composite material may be performed in an oven or kiln.

Step 3) of the process according to the invention may be performed by heat treatment at a temperature ranging from 500° C. to 900° C., preferably 700° C., for a time ranging from 1 hour to 4 hours, preferably 2 hours.

This third step allows the transformation of the zinc salt crystals formed in step 2) into zinc oxide crystals spread over the surface of the diatomaceous earth particles. The zinc oxide is advantageously covalently bonded to the surface of the diatomaceous earth particles.

The heat treatment performed in step 3) may be performed in an oven, in particular with a temperature rise of 190°C/hour up to 700° C., followed by a steady stage at 700° C. for 2 hours before returning to room temperature by switching off the oven. A more gradual temperature rise and a longer steady stage may also be used, without impairing the result.

Step 4) of milling of the microparticles obtained in step 3) to an individual particle size ranging from 10 to 300 nm is performed in aqueous medium in the presence of an agent for stabilizing the electronic charges created during milling. Specifically, the inventors found that milling of these microparticles in aqueous medium without any addition results in reagglomeration or recrystallization of the microparticles obtained during and on conclusion of milling, and thus does not afford the desired particle size. The inventors found, entirely surprisingly, after extensive research, that milling in the presence of an agent for stabilizing the electronic charges created during milling, such as an aqueous alkali metal silicate solution, prevents or at least limits this phenomenon of reagglomeration or recrystallization and thus affords an individual particle size ranging from 10 to 300 nm. Without wishing to be bound by any theory, the inventors believe that when the zinc crystals are milled, an imbalance of electronic charges is created, and that the phenomenon of reagglomeration or recrystallization is due to the attraction of the opposite charges created during milling. According to the inventors'findings, the use of an agent for stabilizing the electronic charges created during milling, such as an aqueous alkali metal silicate solution, allows the new electronic environment created during milling to be stabilized by compensating for the charges that appear. Specifically, in the case of an alkali metal silicate, the alkali metal cations would compensate for the negative charges, and the anions resulting from the dissolution of the alkali metal silicate would compensate for the positive charges.

The alkali metal silicate may be chosen from sodium silicate, lithium silicate and potassium silicate. Preferably, the alkali metal silicate is sodium silicate.

The alkali metal silicate is used in the form of an aqueous solution. The alkali metal silicate is used in an amount suitable for stabilizing the electronic charge imbalance during milling. In the case of sodium silicate, it is advantageously used in an amount of from 3% to 20%, preferably from 5% to 15%, notably from 10% to 15%, the percentages being expressed by weight relative to the weight of the microparticles obtained in step 3) of the process according to the invention.

The milling in step 4) may be performed similarly to that in step 1), notably using the same type of equipment, more particularly a planetary ball mill. A person skilled in the art knows how to adapt the milling conditions so as to obtain the desired particle size.

The microparticles obtained in step 3) are milled in step 4) to an individual particle size ranging from 10 to 300 nm. Preferably, they are milled to an individual particle size ranging from 15 nm to 250 nm. To the best of the inventors'knowledge, such particle sizes were hitherto not accessible for zinc oxide particles grafted onto the surface of diatomaceous earth particles.

Thus, a second subject of the invention is a composite material comprising zinc oxide particles grafted onto the surface of diatomaceous earth particles, the grafted diatomaceous earth particles having an individual particle size ranging from 10 to 300 nm, preferably from 15 to 250 μm. As indicated previously, the term “individual particle size” means that each particle of the material has a particle size falling within the indicated range of values. The particle size is determined by scanning electron microscopy (SEM).

Advantageously, the composite material according to the invention comprises zinc oxide present on the surface of the diatomaceous earth particles at a concentration of 50% to 90% by weight, preferably from 50% to 80% by weight, more preferably from 65% to 75% by weight, even more preferably about 70% by weight relative to the weight of the composite material.

The invention also relates to the use of the composite material according to the invention or prepared via the process according to the invention as an active agent for protecting against UV radiation, notably in cosmetic compositions.

As regards cosmetic compositions, the composite material according to the invention has both high UV-stabilizing efficacy, and numerous advantages due to its size: a very strong limitation of the diffusion of this material into the environment compared to zinc oxide nanoparticles alone, combined with a very homogeneous diffusion in the excipients and a limitation of the visible light reflection effect by the particles, which reduces the major drawback of whitish surface effects on the skin, relative to a material with a larger size, such as unmilled diatomaceous earth.

Another subject of the invention is a cosmetic composition comprising the composite material according to the invention or prepared via the process according to the invention. The cosmetic compositions according to the invention comprise, but are not limited to, formulations for topical application, such as creams, oils, milks, suspensions, sprays, lotions, balms and serums.

The invention will be better understood with the aid of the figures and examples that follow.

### FIGURES

FIG. 1 is a scanning electron microscope image of milled diatomaceous earth particles.

FIG. 2 is a scanning electron microscope image of a composite material according to the invention according to an embodiment using 5% by weight of sodium silicate.

FIG. 3 is a scanning electron microscope image of a composite material according to the invention according to an embodiment using 10% by weight of sodium silicate.

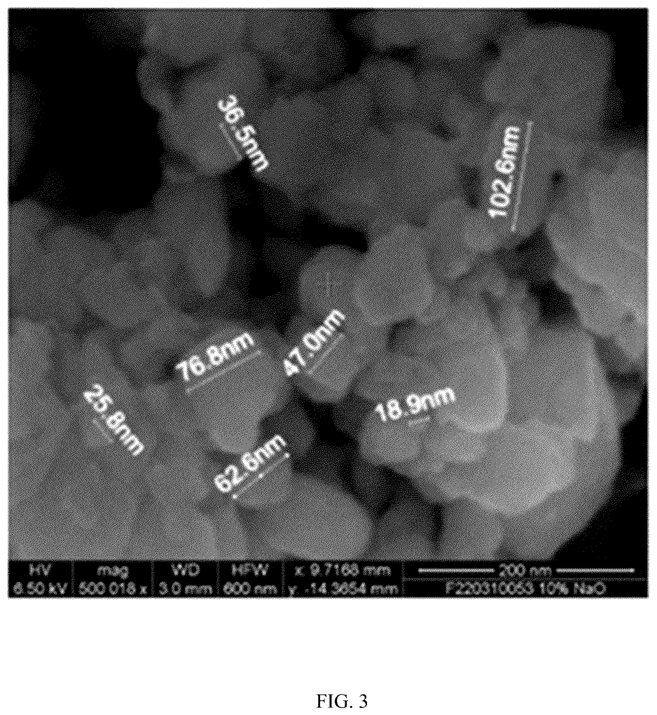

FIG. 4 is a scanning electron microscope image of a composite material obtained by milling in the absence of an electronic-charge stabilizer.

FIG. 5 shows the in vivo UV-stabilizing effect of a composite material according to an embodiment using 10% by weight of sodium silicate 24 h after exposure of the volunteer to sunlight.

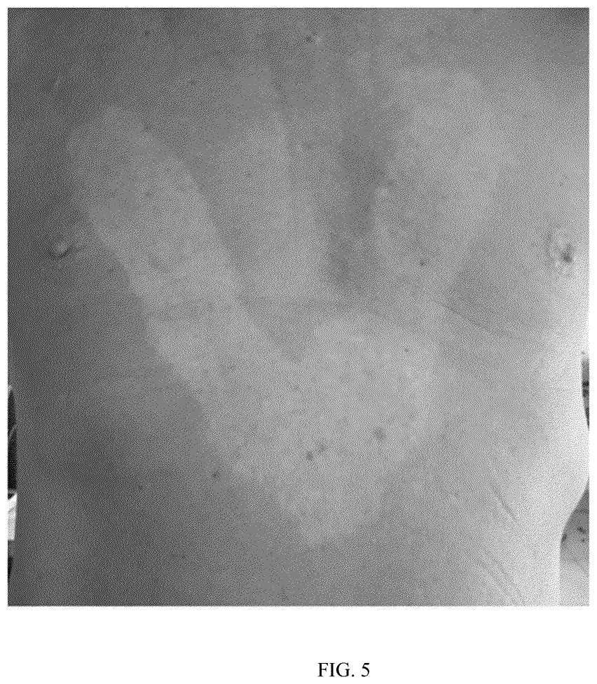

### EXAMPLES

Example 1: Preparation of composite material comprising zinc oxide crystals grafted onto the surface of diatomaceous earth microparticles (steps 1) to 3) of the process according to the invention).

40 grams of diatomaceous earth were milled in a planetary mill (PM100 from Retsch) equipped with a 50 mL bowl with the following parameters:
Milling feedstock volume: 60% of milling bowl capacity;
Milling feedstock particle size. Milling balls with a diameter of 0.5 mm;
Product feedstock: 16% of bowl volume;
Water feedstock: 24% of bowl volume;
Rotational speed: 550 rpm;
Number of milling cycles. 8 cycles of 10 minutes with 10-minute pause periods between each milling phase.

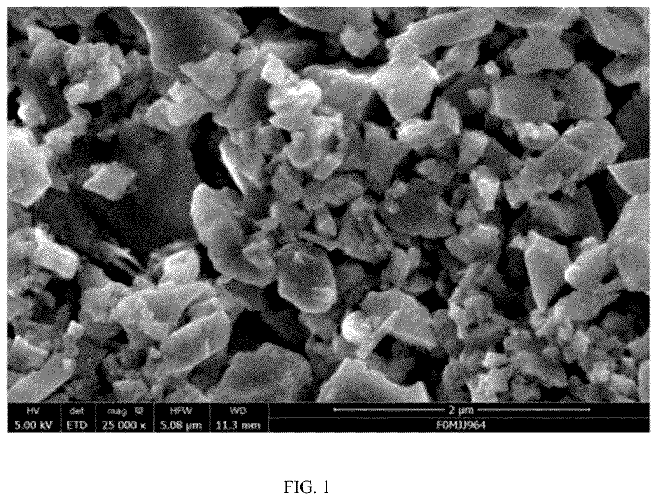

Several 40-gram batches of diatomaceous earth were milled in this manner to recover 1000 grams of diatomaceous earth powder. According to the scanning electron microscopy analyses, this powder shows fairly homogeneous milling of the diatomaceous earth particles below 500 nm (see FIG. 1). The recovered powder was suspended in tap water at a mass concentration of 10 g of powder per liter of water, and 1000 grams of calcium carbonate were then added at a mass concentration of 10 g per liter of water.

In parallel, 13 600 grams of ZnCl₂ were dissolved in tap water to prepare a solution with a concentration of 1 mol/liter.

Hydration of the two suspensions was performed separately for 30 minutes prior to mixing of the two. The two aqueous phases thus obtained were then mixed in a suitable vessel. Stirring of the aqueous mixture thus obtained was performed by means of a paddle mixer at a rotational speed of about 100 rpm and at a temperature of between 15° C. and 25° C., for a period of 48 hours.

The composite obtained on conclusion of mixing of the two aqueous phases was then recovered by a first step of gravitational emptying of the mixing vessel into a recovery tank with a plastic bag. Several decanting phases were performed to eliminate the surface water, and the material thus recovered in the form of a cream was then dried by placing the cream into trays which were then placed in an oven to dry at 90° C. for 24 h.

The powder resulting from this drying process was then transferred into calcination saggars, which were finally placed in the oven for calcination, with a programmed temperature rise of 190° C./hour to 700° C., followed by a steady stage at 700° C. for 2 hours before returning to room temperature by switching off the oven. At the end of this step, about 10 kg of the composite material according to the invention were recovered.

Example 2: Milling the Composite Material Obtained in Example 1 in the Presence of sodium silicate.

10 g of the composite material obtained according to Example 1 were milled in the presence of tap water and an aqueous sodium silicate solution (Sodium Silicate 40% from Arcane Industries, CAS No. 1344-09-8) in a planetary mill equipped with a 50 ml bowl and 0.5 mm milling balls in a proportion of one tenth of the available amount, i.e. about 100 g.

The milling parameters were as follows:
Total cycle time: 8 hours
Parameters: 32 cycles of 10 minutes of milling separated by 5-minute pauses (i.e. 5 h 20 min of effective milling)
Rotational speed: 500 rpm
Wait for the cycle to finish and for the bowl to cool
Rinse with a new 250 μm Retsch sieve with water and a brush to recover the balls. Weigh the water additions
Recovery and storage of the various creams obtained: about 10 g dry equivalent recovered per test.

Two tests with different concentrations of sodium hydroxide silicate were performed:
Test 2-1: 5% by weight of sodium silicate relative to the weight of the composite material, i.e. 1250 mg of 40% sodium silicate solution per 10 g of composite material used;
Test 2-2: 10% by weight of sodium silicate relative to the weight of the composite material, i.e. 2500 mg of 40% sodium silicate solution per 10 g of composite material used.

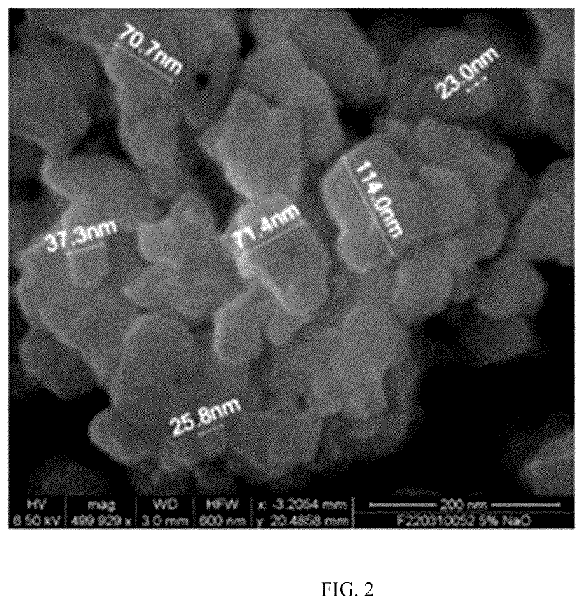

For both tests, the individual particle size ranges from 10 to 300 nm, as shown in FIGS. 1 and 2.

Example 3: Milling the composite material obtained in Example 1 without adding sodium silicate (Comparative Example)

10 g of composite material obtained according to Example 1 were milled in the presence of 20 g of tap water in a planetary mill equipped with a 50 mL bowl and 0.5 mm milling balls in a proportion of one tenth of the available amount, i.e. about 100 g. The milling parameters were as follows:
Total cycle time: 8 hours
Parameters: 32 cycles of 10 minutes of milling separated by 5-minute pauses (i.e. 5 h 20 min of effective milling)
Rotational speed: 500 rpm
Wait for the cycle to finish and for the bowl to cool
Rinse with a new 250 μm Retsch sieve with water and brush to recover the balls. Weigh the water additions
Recover and store the cream obtained: about 10 g dry equivalent recovered.

Example 4: Evaluation of the in vivo UV-stabilizing effect of the material of Example 2, prepared with 10% sodium silicate.

The material of Example 2 prepared with 10% sodium silicate was tested to evaluate its ability to protect human skin against UV radiation (UV-stabilizing effect). The test was performed on a healthy human volunteer with no photosensitivity problems and having type I skin, i.e. skin that always reddens and never tans.

About 1 g of the cream recovered on conclusion of Example 2, with 10% sodium silicate used, was applied in a V-shape to the volunteer's torso and they were then exposed to sunlight from 1 pm to 3 pm at the end of May in Brittany (France).

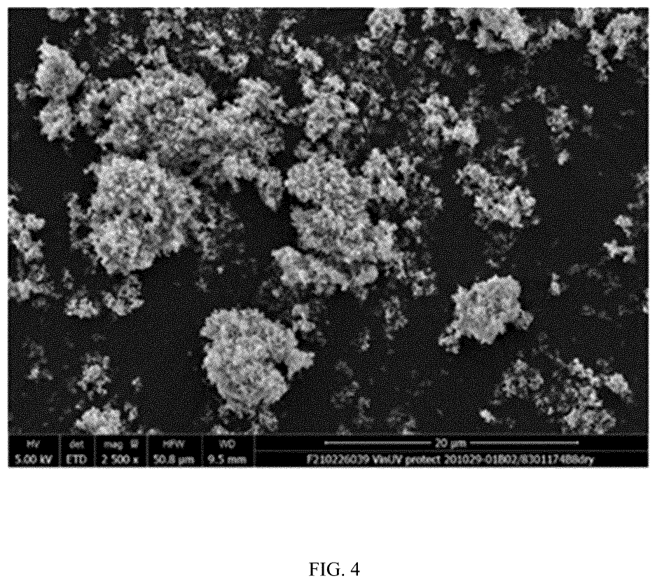

After exposure, the unprotected skin had reddened considerably, while the V-shaped part protected with the material according to the invention remained unchanged. After 24 h, the unprotected skin was still very red and the protected part was unchanged, as may be seen in FIG. 4.

1. A process for preparing a composite material comprising zinc oxide crystals grafted onto the surface of diatomaceous earth particles, comprising the following steps:
1) preparing diatomaceous earth microparticles by milling diatomaceous earth in aqueous medium;
2) grafting zinc salt crystals onto the surface of the diatomaceous earth microparticles;
3) converting said zinc salt crystals grafted onto the surface of the diatomaceous earth microparticles into zinc oxide crystals grafted onto the surface of the diatomaceous earth microparticles;
4) milling the microparticles obtained in step 3) in aqueous medium to an individual particle size ranging from 10 to 300 nm, this milling being performed in the presence of an agent for stabilizing the electronic charges created during milling.

2. The process as claimed in claim 1, in which the milling step 1 is performed using a planetary mill.

3. The process as claimed in claim 1, in which step 2 comprises the following steps:
a) placing in aqueous suspension diatomaceous earth particles obtained in step 1) with calcium carbonate;
b) placing the mixture obtained at the end of step a) in contact with an aqueous suspension of zinc chloride;
c) stirring the mixture obtained at the end of step b) at a temperature ranging from 5° C. to 40° C., preferably from 15° C. to 25° C., for a period ranging from 24 to 72 hours, preferably 48 hours;
d) recovering the composite material obtained at the end of step c), notably by sedimentation or centrifugation;
e) drying the composite material at a temperature ranging from 50° C. to 90° C., preferably 70° C., for a period ranging from 12 to 48 hours, preferably 24 hours.

4. The process as claimed in claim 1, in which step 3 is performed by heat treatment at a temperature ranging from 500° C. to 900° C., preferably 700° C., for a time ranging from 1 to 4 hours, preferably 2 hours.

5. The process as claimed in claim 1, in which the agent for stabilizing the electronic charges created during milling is an aqueous alkali metal silicate solution.

6. The process as claimed in claim 5, in which the alkali metal silicate solution is a sodium silicate solution.

7. A composite material comprising zinc oxide crystals grafted onto the surface of diatomaceous earth particles, said grafted diatomaceous earth particles having an individual particle size ranging from 10 to 300 nm.

8. The composite material as claimed in claim 7, in which zinc oxide is present on the surface of the diatomaceous earth particles at a concentration of from 50% to 80% by weight, preferably from 65% to 75% by weight, even more preferably about 70% by weight relative to the weight of the composite material.

9. The composite material as defined in claim 7 or prepared as claimed in claim 1, for use as an active agent for protecting against UV radiation, notably in cosmetic compositions.

10. A cosmetic composition comprising the composite material as defined in claim 7, or prepared as claimed in claim 1.
